(19) United States
(12) Patent Application Publication
(10) Pub. No.: US 2004/0138822 A1
(43) Pub. Date: Jul. 15, 2004

(54) Method and system for managing batches of immunocompetent cells collected from human or animal subjects for deferred use, and related therapy methods

(76) Inventor: Patrick Rambaud, Montreal (CA)

Correspondence Address: YOUNG & THOMPSON, 745 SOUTH 23RD STREET 2ND FLOOR, ARLINGTON, VA, 22202

(21) Appl. No.: 10/687,636

(22) Filed: Oct. 20, 2003

Related U.S. Application Data
(63) Continuation-in-part of application No. 09/685,961, filed on Oct. 16, 2000, now abandoned.

(30) Foreign Application Priority Data
Jan. 21, 2000 (FR) 00 00804

(51) Int. Cl. G06F 19/00; G01N 33/48; G01N 33/50; C12N 5/08

(52) U.S. Cl. 702/19; 435/366

(57) ABSTRACT

A method for managing batches of immunocompetent cells collected from human or animal subjects for deferred use, comprising for each of said human or animal subjects:

conditioning and preserving successively collected batches of immunocompetent cells, into one or more storage centers, and

constituting and enhancing from collected batches a personal library of immunocompetent cells, said personal library cumulating a sum of immunity information stored in the collected immunocompetent cells,

This method further comprises:

gathering information characteristic of the status of said human or animal subject, effected before or during the immunocompetent cells collection, and

processing said characteristic information for determining parameters of a deferred-use protocol for immunocompetent cells from said human or animal subject's personal library.

[Continuation-In-Part of Ser. No. 09/685,961 filed Oct. 16, 2000]

### OBJECT OF THE INVENTION

The present invention relates to a method for managing batches of immunocompetent cells, particularly leucocytes, lymphocytes, monocytes, for deferred use. It also concerns a management system for using the method according to the invention, a method and system for determining parameters of a protocol for a deferred use of immunocompetent cells, implemented in the management system according to the invention, and therapy methods wherein said management method can be implemented.

### TECHNICAL BACKGROUND

Scientific and clinical works have demonstrated the therapeutic qualities of auto-use of lymphocyte and monocyte derivatives which helps, in particular, to increase cell immunity.

A promising application of this therapeutic method relates to the possibility of strengthening the immunity of a patient at a time in his life when this strengthening proves to be necessary or vital, or to maintain this immunity throughout his life.

However, a significant difficulty to be overcome lies in the availability of immunocompetent cells of a patient over periods of time which could be between several months and several decades. Techniques of cryogenic storage for the future which are widely used in several fields of human and animal biology are already known. In particular, banks have been established for preservation and storage of biological elements.

Document WO8904168 discloses a method for isolating and preserving hematopoïetic cells from fetal and neonatal blood. This method is aimed to a therapeutic use of fetal and neonatal cells for hematopoïetic reconstitution or in gene therapy, and can be implemented for cryo-preserving fetal or neonatal blood cells aimed to autologous reconstitution.

The immunocompetent cells (lymphocytes, phagocyte cells, monocytes, macrophages) play a leading role in the immune system. In particular, lymphocytes store information during life and are the support of memory for humoral and cellular immunity. These immunocompetent cells constitute in fact a library, in particular a lymphocyte library, which is enhanced during life, when the body meets foreign organisms, during viral, parasitic or bacterial infections. By means of this “immunity library”, the body can minimize the impact of the infections during life. The action mechanism of the immune system is already known. Information are stored in the walls of lymphocytes, as illustrated by the transfer factor and reported by numerous publications. This mechanism also contributes to the defense against malignant cells.

First, this memory is partially being erased with time, as shown by the requirement to achieve vaccination confirmations for preserving an efficient protection. Concerning the humoral immunity, the antibody ratios decrease, quickly for the IgM, more slowly for the IgG and the IgA.

Moreover, errors are introduced with time and the immunity becomes usually less efficient with years. Because of this degradation, infections as flu are far more dangerous for aged persons. Furthermore, it would be particularly interesting to preserve information acquired along a whole life.

Document WO9953030 discloses a method for managing batches of immunocompetent cells, comprising for a human subject:

conditioning and storing batches of immunocompetent cells in one or more storage centers,

constituting and enhancing a personal library of immunocompetent cells from successively collected batches, said personal library preserving a sum of immunity information stored the collected immunocompetent cells, and

in response to a request for treatment of a said human subject:

processing all or part of said immunity information cumulated in said personal library, and

localizing one or more stored batches of immunocompetent cells, and then transferring this or these batch(es) to a requesting cellular-treatment center.

Such a process thus provides patients with the guarantee of storage of their lymphocytes in the long term, with the prospect of having them available at any time for, inter alia, strengthening of their immune system. It therefore becomes possible to give back to people their former immunity and to transmit a cell immunity under rational and reliable management conditions, and also to have access to their corresponding genetic code at the time of collection of the blood.

But besides the need for preserving immunocompetent cells in view of a deferred use, main questions concerning the definition of a suitable protocol for this deferred use are raised. In fact, it was shown that a mere re-injection of immunocompetent cells previously collected on a human subject could result in potentially serious immunity problems. Moreover, it was shown that the quality and potential efficiency of collected immunocompetent cells could be highly dependant on the human subject's general status of health, with adverse consequences for the performance of the present methods.

### SUMMARY OF THE INVENTION

The object of the present invention is to remedy these disadvantages by proposing a process for management of batches of immunocompetent cells collected and preserved for deferred use, which fully integrates, for human subjects and extensively for animal subjects, the dimension of identity and status of health.

This object is achieved by a method for managing batches of immunocompetent cells collected from human or animal subjects for deferred use, comprising for each of said human or animal subjects the following steps:

conditioning and preserving successively collected batches of immunocompetent cells, into one or more storage centers, and

constituting and enhancing from collected batches a personal library of immunocompetent cells, said personal library cumulating a sum of immunity information stored in the walls of the collected immunocompetent cells,

during successive collections or batches, gathering information characteristic of the status of health and/or the psychological status of said human or animal subject, said status-characterizing information being obtained by processing measurements made on samples of blood and/or fluid and secretions and/or hair collected said human or animal subject, said status-characterizing information gathering being effected before or during the immunocompetent cells collection,

processing said status-characterizing information for determining the subject's identity data,

storing, all along said steps, the subject's identity data into a cell management database,

upon a request for re-use from a cell treatment entity, performing an identification of the personal batches of cells by consulting said cell management database, and receiving from said cell management database said subject's identity data obtained by successive status-characterizing information processing,

determining parameters of a deferred-use protocol of said batches of immunocompetent cells, by processing said successively collected subject's identity data, and

providing said cell treatment entity with said identified personal batches of cells and with said deferred-use protocol parameters.

With the method according to the invention, information characterizing the human or animal subject's status such as the state of health and psychological status can be processed both for determining the opportunity of collecting immunocompetent cells and for selecting parameters of a deferred-use protocol.

By means of the successively collected batches of immunocompetent cells from a person, a personal library is therefore constituted for said person. This personal library, which gathers immunity information stored in the walls of the collected immunocompetent cells, can be accessed on demand, when required for a therapeutic protocol, in order to provide with pertinent immunity concerning the patient.

The status-characterizing information are preferably obtained by processing a blood sample collected from the human or animal subject. They may comprise bioelectronic information resulting from processing respective measures of pH, oxidation-reduction potential Rh2 and resistivity of blood previously collected on said human or animal subject according to the Vincent's bioelectronic method.

The status-characterizing information is processed to determine a subject's identity data, for example by extracting from said status-characterizing information relevant data on personal immunity history and data. The subject's identity data may include immunity-related data, historical and clinical data on previous diseases, treatments and therapeutic protocols experienced by said subject.

The deferred-use protocol comprises, as a way of non limitative example, a plurality of steps or sequences for retrieving and reconditioning previously stored and preserved batches of immunocompetent cells in view of using said cells for re-injection, and steps for processing the subject's identity data in order to determine for example what type of immunocompetent cells must or can be used with regards to the object of said deferred-use for a given patient, and how and when such selected immunocompetent cells have to be injected in relation with the patient's health status. This protocol can implement deferred-use parameters such as qualitative and quantitative data on the reconditioned immunocompetent cells, and physiological parameters related to the subject's health status in view of cell auto-use.

The status-characterizing information may also comprise information obtained by processing sensible crystallization images of blood previously collected on said human or animal subject, and/or information obtained from a capillary study on elements of said human or animal subject's hair.

Diagnostic methods implementing sensible crystallization images of blood are known for a long time, for example from works published by E. Pfeiffer in “Kristalle. Orient-Occident” Verlag, Stuttgart (1930). Variations of the growth shapes of dendritic crystals of cupric chloride have been studied depending on the disease of a patient whose blood has been added in low quantity to aqueous solutions.

The status-characterizing information and the immunity information stored in the immunocompetent cells of said human or animal subjects are advantageously entered into an expert system used for determining parameters for deferred-use protocols. This expert system can be arranged for providing an interpretation of said status-characterizing information and said immunity information with respect to a particular gene.

The process of the status-characterizing information is arranged for determining respective optimal proportions of different immunocompetent cells in view of their deferred use, and, for example, can provide with a determination of an optimal ratio between lymphocytes T4 and T8 in view of their deferred use.

When the method according to the invention is implemented in a therapeutic protocol including re-injecting lymphocytes on a human or animal subject, the previously collected and preserved immunocompetent cells can be submitted to an ex-vivo process before being re-injected. The method according to the invention can also be implemented in a therapeutic protocol including re-injecting lymphocytes T with a specific cytotoxic activity after ex-vivo expansion, or in a gene therapy protocol.

The therapy protocol wherein the management method according to the invention is implemented generally includes a step for checking the harmlessness of lymphocytes before re-injection. This checking step comprises a test of the oxidative stress of the lymphocytes before re-injection, during which said lymphocytes are aggressed by free radicals. In an evolutionary test for a period of roughly 3 to 6 months, ill-stored lymphocytes when submitted to free radicals generate a quantifiable oxidative stress.

The oxidative stress test can also advantageously used to check various therapy ways for an ex vivo processing and their matching with the human or animal subject concerned by said therapy. Thus, an excessively aggressive lymphocyte product will produce a great amount of free radicals. In the case of an ex vivo processing including a cytotoxic processing, this test enables to choose the most adequate therapy for a patient.

Another promising way of implementation for the management method according to the invention relates to therapy protocols including an ex vivo processing between lymphocytes and a vaccine before re-injection. No production of antibodies has been observed.

The management method according to the invention can further be implemented in a therapy protocol including an ex vivo processing for an allergic desensitization of lymphocytes before re-injection. The product obtained from said protocol is then mixed with lymphocytes stored before re-injection.

In another implementation of the management method according to the invention, the related therapy protocol includes re-injecting lymphocytes into a patient's body by the lymphatic way. The aim is reduce the prescribed quantities in view of a better tolerance by said patient and a greater reaction speed.

The management method according to the invention can also be implemented in a therapy protocol for transfusing blood from a donor to a receiver, said protocol including substituting lymphocytes from the donor by lymphocytes from the receiver.

The management method according to the invention can further comprise, before the step of cryo-preservation of a batch of immunocompetent cells, an initial step for cryogenizing said batch arranged for causing the antibodies initially present in said batch to be annihilated. A step for checking the annihilation of other antibodies within the batch of immunocompetent cells can also be provided.

In a particular embodiment of the method according to the invention, said method further comprises, during a sequence for conditioning a batch of immunocompetent cells previously sampled, a step of immunomagnetic selection for purified lymphocytes or monocytes.

According to another aspect of the invention, there is proposed a system for managing batches of immunocompetent cells collected from human or animal subjects for their deferred use, said system comprising for each of said human or animal subjects:

means for conditioning and preserving batches of immunocompetent cells successively collected, into one or more storage centers,

means for constituting and enhancing from said collected batches a personal library of immunocompetent cells, said personal library cumulating a sum of immunity information stored in the walls of collected immunocompetent cells,

means for gathering, during successive collections of batches, information that are characteristic of said human or animal subject's status of health and/or psychological status, before or during immunocompetent cells collection, said status characterizing information being obtained by processing measurements made on samples of blood and/or fluid and secretions and/or hair collected on said human or animal subject,

means for processing said status-characterizing information in view of determining said subject's identity data,

means for storing said subject's identity data successively determined into a cell management database,

means for performing, upon a request for re-use from a cell treatment entity, an identification of the personal batches of cells to including means for consulting said cell management database,

means for determining parameters of a deferred-use protocol for said batches of immunocompetent cells from said human or animal subject's personal library, by processing said successively collected subject's identity data, and

means for providing said cell treatment entity with said identified personal batches of cells and with said determined deferred-use protocol parameters.

According to still another aspect of the invention, there is proposed a therapy method comprising the step of re-injecting immunocompetent cells in the body of a human or animal subject, said immunocompetent cells having been previously collected during one or more collecting steps from said human or animal subject, and then conditioned, preserved and stored, and constituting a personal library cumulating a sum of immunity information stored in said collected immunocompetent cells.

Said therapy method is characterized in that it is controlled by a deferred-use protocol for said immunocompetent cells which includes parameters obtained by processing immunity information stored in said immunocompetent cells and information characteristic of the status of said human or animal subject, said characteristic information having been gathered before or during the one or more collecting steps.

In a therapy protocol including re-injecting lymphocytes on a human or animal subject, the previously collected and preserved immunocompetent cells are submitted to an ex-vivo process before being re-injected.

The therapy protocol can include re-injecting lymphocytes T with a specific cytotoxic activity after ex-vivo expansion.

According to still another aspect of the invention, there is proposed a method for determining parameters of a protocol for a deferred use of immunocompetent cells from a human or animal subject's personal library, said personal library cumulating a sum of immunity information stored in the immunocompetent cells successively collected and conditioned under the form of batches preserved in one or more storage centers, characterized in that said method comprises:

measuring physical and/or biological characteristics done on samples of fluid and/or hair from said human or animal subject before or during the collection of immunocompetent cells,

collecting information characteristic of said human or animal subject's status resulting from said measurements,

processing said characteristic information in an information system for determining parameters of said deferred-use protocol, and

storing said processed information in a cell management data base.

There is also proposed a system for determining parameters of a protocol for a deferred use of immunocompetent cells from a human or animal subject's personal library, said personal library cumulating a sum of immunity information stored in the immunocompetent cells successively collected and conditioned under the form of batches preserved in one or more storage centers, characterized in that said system comprises:

means for measuring physical and/or biological characteristics done on samples of fluid and/or hair from said human or animal subject before or during the collection of immunocompetent cells,

means for collecting information characteristic of said human or animal subject's status resulting from said measurements,

means for processing said characteristic information in an information system to determine parameters of said deferred-use protocol, and

means for storing said processed information in a cell management data base.

### DETAILED DESCRIPTION

Other details and advantages of the invention will also become apparent in the description below. Regarding the attached drawings, given as non-limiting examples:

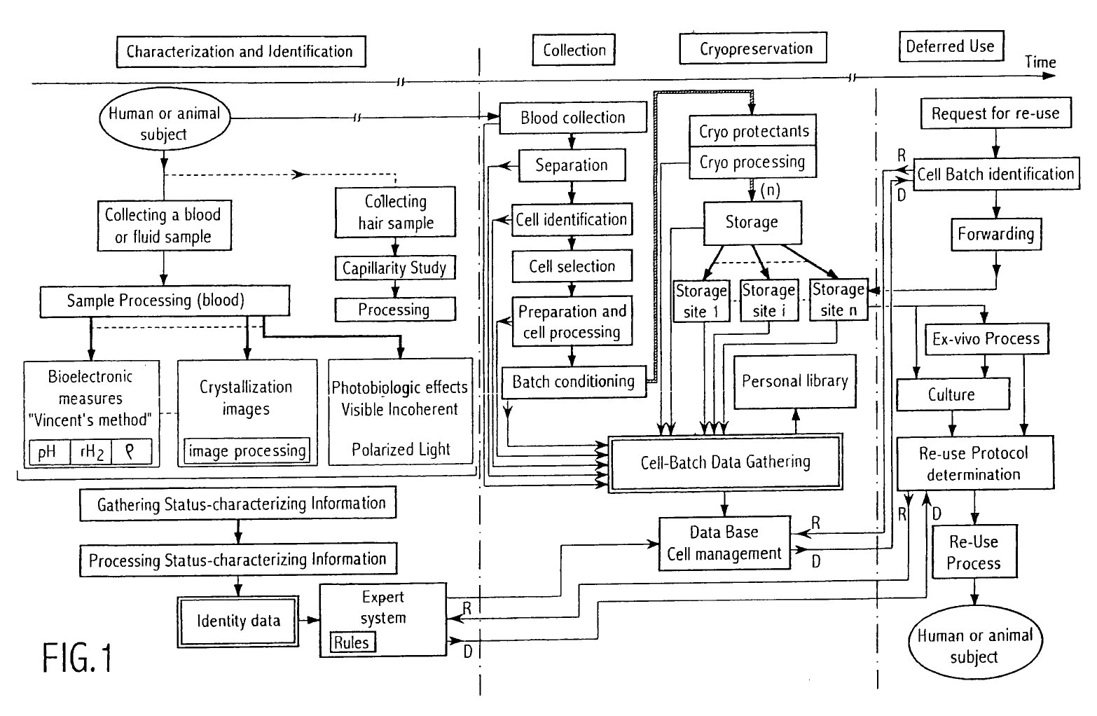

FIG. 1 is a block diagram of the management method according to the invention; and

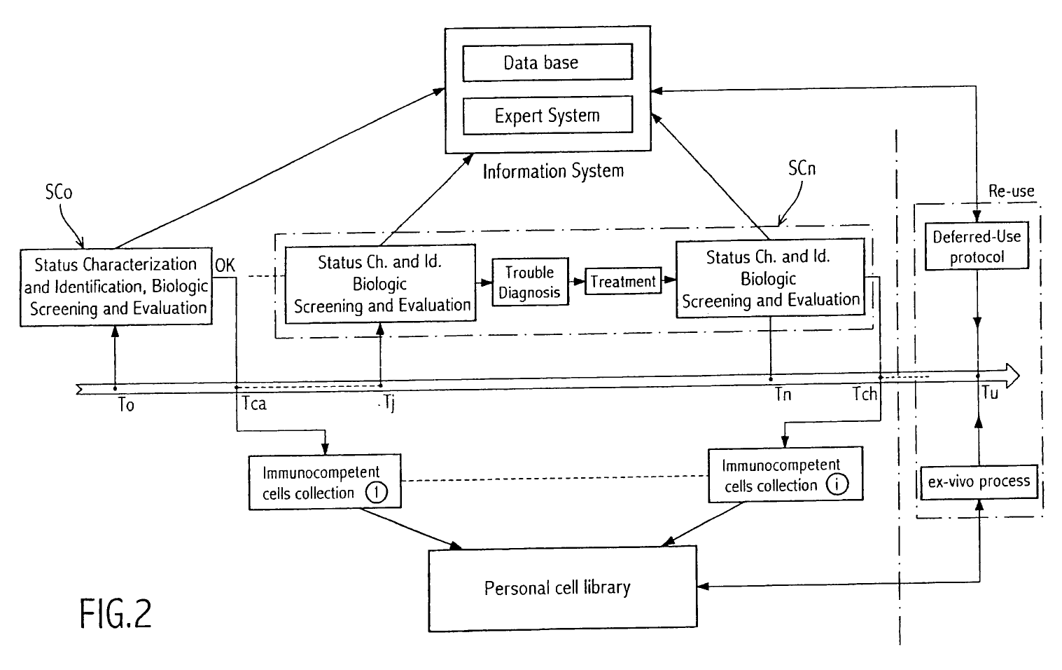

FIG. 2 is a time-diagram featuring main steps of the management method according to the invention effected for a human or animal subject.

The management method according to the method generally includes, with reference to FIG. 1, a first stage of characterization and identification of the human or animal subject's status, successive stages of collection of immunocompetent cells from said subject followed by stages of cryo-preservation and storage of batches of cells, and by one or more stages of deferred use of preserved cells

During the characterization and identification stage, samples of blood and of other fluids and secretions like saliva or urine are collected from the human or animal subject. Samples of hair can also be collected. The blood sample can be processed by any suitable biologic method for providing information characterizing the effective identity.

For example, the Vincent's bioelectronic method, that includes a measurement of the pH, a measurement of the oxidation-reduction potential rH2, and a measurement of the resistivity ρ of the blood sample, provides an interesting characterization of the subject's present status of health.

This bioelectronic method, which is known for a long time, is disclosed for example in the book “Bioelectronic according to Vincent and Acid-Base Household in Theory and Pratice”, Haug Verlag, Heidelberg, 1975, by H. Elmau, and in the book “Theory and Pratice of the Bio-Electronic‘Vincent’”, SIBEV, 1975, by L. Rougon.

Moreover, the processing and interpretation of sensible crystallization images of blood can provide with other status-characterizing information concerning the subject's status of health with precise data on various physiologic troubles.

Status-characterizing information provides by either a bio-electronic process or a sensible crystallization image process, can be corroborated by information processed from a capillarity study on a hair sample.

The whole status-characterizing information are gathered and processed to generate identity data that can be entered into an expert system for which specific rules are implemented.

Following the above-described characterization and identification stage, a collection stage is effected provided that identity data meet physiologic requirements for allowing said collection stage. Blood is collected and separated to get various component cells such as lymphocytes and monocytes. The various cells obtained by separation are identified, prepared and if necessary processed and fractionated in a plurality of n batches. These n batches of immunocompetent cells are then conditioned, cryo-processed and stored into various storage sites 1, . . . ,i, . . . ,n located for ensuring the best integrity and safety for said batches of cells.

It has to be noted that a method of immunomagnetic selection for the purified lymphocytes and monocytes can be used. Furthermore, cryo-protectors can advantageously be used during the cryo-preservation of the immunocompetent cells.

The immunocompetent cells are preferably preserved at a temperature comprised between −80° C. and −120° C. Preservation in liquid nitrogen at −196° C. often raises problems of infrastructure and logistic.

The deferred use of lymphocytes can raise problems if antibodies are preserved because, along the time, the concerned subject or patient may be submitted to a reaction against his or its own antibodies and to a reject of said antibodies. An initial step of cryogenization can be provided in order to annihilate antibodies within a batch of immunocompetent cells. Before reusing this batch of cells, a step for checking the effective annihilation of said antibodies is provided. This checking step can implement known techniques for testing antibodies in batches of cells that have been previously cryo-preserved.

It is important to ensure the structural and functional integrity of the cryo-preserved cells, in order to guarantee their reliability in view of a deferred use.

The batches of immunocompetent cells or haematopoietic system components can thus be stored over widely varying periods, which can range from a few days to several decades, provided that proper storage of the haematopoietic-system components in the long term is guaranteed. Furthermore, the principle of not storing all the batches of one patient at the same site contributes substantially to the security of the supply.

All along these processing sequences, data related to every elementary step are collected, gathered and stored into a cell management database that also receives data from the expert system including the subject's identity data. The stored immunocompetent cells belonging to a human or animal subject constitute his or its personal immunity library that is linked by the cell management database that can be located within a management center controlling the plurality of storage sites and one or more preparation and cryo-preservation centers.

When the management center receives a request from a cell treatment entity, for re-use to the benefit of a human or animal subject, an identification of the personal batches of cells is effected by consulting (R) the cell management database and receiving (D) from said database identification data related to an appropriate batch of cells which is forwarded from a storage site towards a center specifically equipped for deferred re-use.

The cell treatment entity means a medical center, hospital or private structure that is habilitated to process previously cryo-preserved human or animal cells and to re-use said personal cells in a therapeutic process for a patient. This cell treatment entity can also be equipped to collect immunocompetent cells and/or to condition and preserve collected batches of cells.

By interrogation of the database, a batch belonging to this subject is determined and located in one of the storage sites. After location and cell identification, the batch in question is forwarded by express transport to the cell treatment center which can also be the center in which the initial collection was made.

Said batch is the processed to room temperature and immunocompetent cells are put in culture and/or submitted to an ex-vivo process. Parameters of a protocol of re-use are determined by requesting identity data from the cell management database and processing said identity data to determine for example optimal ratio between lymphocytes T4 and T8 for re-injection. In view of a deferred-use therapeutic process for a patient, the cell treatment entity is therefore provided with one or several identified batches of cells from said patient and with determined re-use or deferred-use protocol parameters.

Said re-use or deferred-use protocol is then implemented in a re-use process applied to the human or animal subject.

The management method according to the invention allows the constitution all along a human or animal subject's life of both a personal “cell library” and of a personal database containing data resulting from successive characterization stages and data generated by use of the expert system, as illustrated by FIG. 2. As a way of example, at an instant To, a human or animal subject is submitted to a status characterization process SCo that provides with information characterizing the subject's physiologic identity and state of health. If this characterization stage results in a correct evaluation, a stage for collecting immunocompetent cells is then effected at instant Tco on the human or animal subject.

The expert system embodied in a management system according to the invention can implement known conventional structures of expert systems in the field of biology and biological analysis. The status-characterizing information corresponding to a subject or patient are entered into the expert system, in the form of biological items to which a set of rules stored in a knowledge base is applied to generate the subject's identity data.

For example, it can be referred to U.S. Pat. No. 5,694,950 disclosing a method and system for use in treating a patient with immunosuppresants using whole blood level criteria to prevent an adverse immune response, that implements an expert system.

At a further instant Tj, another status-characterization stage is effected on the human or animal subject and this characterization stage results in data revealing a physiologic trouble preventing from any cell collection. A therapeutic treatment can be proposed in order to remedy the diagnosed trouble and another characterization stage is further effected at instant Tn until getting a correct evaluation allowing a collection stage i of immunocompetent cells.

Data resulting from the successive status-characterization stage are gathered, processed in the expert system and stored in cell management database, while collected immunocompetent cells enhance the subject's personal cell library.

When a re-use process of immunocompetent cells is prescribed for a human or animal subject, a protocol of deferred-use is determined using data stored in the database with, for example, optimal proportions between each type of cells. Selected immunocompetent cells are then extracted from the personal cell library and, if necessary, processed ex-vivo. When these immunocompetent cells are ready for use, an re-use process according to the determined personal protocol is effected at instant Tu.

It should be noted that each patient or subject following such a program generally has available a stock of batches of immunocompetent cells or haematopoietic-system components, which enables him, for example, to spread successive auto-uses, for example in the form of auto-injections, over a period of time, with the aims of strengthening the immune system or gene or other therapy, or also to use them massively if the stock of haematopoietic-system components made up in this way is required.

The management method according to the invention is preferably implemented in the form of a software installed on management and data processing systems, which may be located in batch management centers and be connected to all the data processing sites located within the cytopheresis, express logistics and storage centers.

It has to be noted that a management system according to the invention can be entirely automated, from the collection of information characteristic of the physical and/or biological status of a subject, through the preservation and storage of immunocompetent cells, up to the determination of protocols for deferred-use of said immunocompetent cells. The protocol determination process can be advantageously implemented in an expert system processing past experimental and clinical data related to deferred-use cumulated practice. For example, a deferred-use protocol may comprise as a way of non-limitative example, an optimal time schedule indicating the proposed dates for deferred use depending on collected personal parameters and therapeutic indications for re-use, and biological and technical indications required for cell processing before re-use.

In a management system according to the invention, a personal library cumulating a sum of immunity information stored in the collected immunocompetent cells, can be managed for each human or animal subject. Personal characteristic Information collected during the biological and physical analysis sequences can be stored, either close to the corresponding personal immunocompetent cells, for example in an electronic chip or microcircuit within or close to the preservation devices, or in global storage units remote from the cell storage and preservation units and accessible through communication networks. Hybrid and redundant solutions combining close storage of both immunocompetent cells and related information and remote information storage can also be provided.

Besides the numerous applications contemplated in the field of human therapy, the method according to the invention can also be implemented in the field of mass animal production, particularly as an alternative to the antibiotic therapy, for racehorses in order to provide them with an immunity protection, and for pet animals as an anti-aging factor and for its action on the immunity system.

The invention is of course not limited to the examples which have just been described, and several modifications may be made to these examples without going beyond the context of the invention. In the context of the collection and re-use phases, additional technical stages can thus be provided, according to medical requirements and safety constraints. The tools of communication used between the various operational and management centers can be of any nature.

Furthermore, the data gathered in the context of the management process according to the invention may advantageously be processed for statistical purposes, with applications in the field of prevention and insurance.

1. A method for managing batches of immunocompetent cells collected from human or animal subjects for deferred use, comprising for each of said human or animal subjects the following steps:
conditioning and preserving successively collected batches of immunocompetent cells, into one or more storage centers, and
constituting and enhancing from collected batches a personal library of immunocompetent cells, said personal library cumulating a sum of immunity information stored in the walls of the collected immunocompetent cells,
during successive collections or batches, gathering information characteristic of the status of health and/or the psychological status of said human or animal subject, said status-characterizing information being obtained by processing measurements made on samples of blood and/or fluid and secretions and/or hair collected said human or animal subject, said status-characterizing information gathering being effected before or during the immunocompetent cells collection,
processing said status-characterizing information for determining the subject's identity data,
storing, all along said steps, the subject's identity data into a cell management database,
upon a request for re-use from a cell treatment entity, performing an identification of the personal batches of cells by consulting said cell management database, and receiving from said cell management database said subject's identity data obtained by successive status-characterizing information processing,
determining parameters of a deferred-use protocol of said batches of immunocompetent cells, by processing said successively collected subject's identity data, and
providing said cell treatment entity with said identified personal batches of cells and with said deferred-use protocol parameters.

2. The method according to claim 1, characterized in that the status-characterizing information comprise bioelectronic information resulting from processing respective measures of pH, oxidation-reduction potential Rh2 and resistivity ρ of blood previously collected on said human or animal subject (Vincent's biolectronic method).

3. The method according to claim 1, characterized in that status-characterizing information comprise information obtained by processing sensible crystallization images of blood previously collected on said human or animal subject.

4. The method according to claim 1, characterized in that the status-characterizing information and the immunity information stored in the immunocompetent cells of said human or animal subjects are entered into an expert system used for determining parameters for deferred-use protocols.

5. The method according to claim 4, characterised in that said expert system is arranged for providing an interpretation of said status-characterizing information and said immunity information with respect to a particular gene.

6. The method according to claim 1, implemented in a therapeutic protocol including re-injecting lymphocytes on a human or animal subject, characterised in that the previously collected and preserved immunocompetent cells are submitted to an ex-vivo process before being re-injected.

7. The method according to claim 6, implemented in a therapeutic protocol including re-injecting lymphocytes T with a specific cytotoxic activity after ex-vivo expansion.

8. The method according to claim 6, implemented in a therapy protocol including a step for checking the harmlessness of the lymphocytes before re-injection.

9. The method according to claim 8, implemented in a therapy protocol including a checking step comprising a test of the oxidative stress of the lymphocytes before re-injection, wherein said lymphocytes are aggressed by free radicals.

10. The method according to claim 9, implemented in a therapy protocol including an oxidative stress test for checking various therapy ways for an ex vivo processing and suitability of said therapy ways with the concerned human or animal subject.

11. The method according to claim 6, implemented in a therapy protocol including an ex vivo processing between lymphocytes and a vaccine before re-injection.

12. The method according to claim 6, implemented in a therapy protocol including an ex vivo processing and an allergic desensitization of the lymphocytes before re-injection.

13. The method according to claim 6, implemented in a therapy protocol including a step for re-injecting lymphocytes by the lymphatic way.

14. The method according to claim 6, implemented in a therapy protocol for transfusing blood from a donor to a receiver, said protocol including substituting lymphocytes from said donor by lymphocytes from said receiver.

15. The method according to claim 1, implemented in a gene therapy protocol.

16. The method according to claim 1, characterized in that it further comprises, before the step for cryo-preserving a batch of immunocompetent cells, a step of cryogenizing said batch in view of annihilating antibodies present within said batch.

17. The method according to claim 20, characterized in that it further comprises, before any re-use of a batch of immunocompetent cells previously collected, a step for checking the annihilation of the antibodies within said batch.

18. The method according to claim 1, characterized in that it further comprises, during conditioning a batch of immunocompetent cells previously collected, a step for immunomagnetically selecting purified lymphocytes or monocytes.

19. A system for managing batches of immunocompetent cells collected from human or animal subjects for their deferred use, said system comprising for each of said human or animal subjects:
means for conditioning and preserving batches of immunocompetent cells successively collected, into one or more storage centers,
means for constituting and enhancing from said collected batches a personal library of immunocompetent cells, said personal library cumulating a sum of immunity information stored in the walls of collected immunocompetent cells,
means for gathering, during successive collections of batches, information that are characteristic of said human or animal subject's status of health and/or psychological status, before or during immunocompetent cells collection, said status characterizing information being obtained by processing measurements made on samples of blood and/or fluid and secretions and/or hair collected on said human or animal subject,
means for processing said status-characterizing information in view of determining said subject's identity data,
means for storing said subject's identity data successively determined into a cell management database,
means for performing, upon a request for re-use from a cell treatment entity, an identification of the personal batches of cells to including means for consulting said cell management database,
means for determining parameters of a deferred-use protocol for said batches of immunocompetent cells from said human or animal subject's personal library, by processing said successively collected subject's identity data, and
means for providing said cell treatment entity with said identified personal batches of cells and with said determined deferred-use protocol parameters.

20. The system according to claim 19, characterized in that it further comprises means for getting status-characterizing by processing a blood sample collected on said human or animal subject.

21. The system according to claim 20, characterized in that it further comprises means for getting bio-electronic information by processing respective measures of the pH, the oxidation-reduction potential and the resistivity of blood previously collected on said human or animal subject.

22. The system according to claim 19, characterized in that it further comprises means for getting information from a capillary study on elements of said human or animal subject's hair system.

23. The system according to claim 19, characterized in that it further comprises means for controlling and enhancing an expert system from information characteristic of the status of human or animal subject's and from immunity information stored in said human or animal subject's immunocompetent cells, in view of determining parameters for deferred-use protocols.

24. The system according to claim 23, characterized in that it further comprises means for providing an interpretation of said human or animal subject's status-characterizing information and said immunity information, with respect of a particular gene.

25. A method for determining parameters of a protocol for a deferred use of immunocompetent cells from a human or animal subject's personal library, said personal library cumulating a sum of immunity information stored in the walls of the immunocompetent cells successively collected and conditioned under the form of batches preserved in one or more storage centers, characterized in that said method comprises:
measuring physical and/or biological characteristics done on samples of fluid and/or hair from said human or animal subject before or during the collection of immunocompetent cells,
collecting information characteristic of the status of health and/or the physiological status of said human or animal subject's status resulting from said measurements, said status characterizing information being obtained by processing measurements made on samples of blood and/or fluid and secretions and/or collected on said human or animal subject,
processing said characteristic information in an information system for determining parameters of said deferred-use protocol, and
storing said processed information in a cell management data base.

26. A system for determining parameters of a protocol for a deferred use immunocompetent cells from a human or animal subject's personal library, said personal library cumulating a sum of immunity information stored in the walls of the immunocompetent cells successively collected and conditioned under the form of batches preserved in one or more storage centers, characterized in that said system comprises:
means for measuring physical and/or biological characteristics done on samples of fluid and/or hair from said human or animal subject before or during the collection of immunocompetent cells,
means for collecting information characteristic of the status of health and/or the psychological status of said human or animal subject's status resulting from said measurements, said status characterizing information being obtained by processing measurements made on samples of blood and/or fluid secretions and/or hair collected on said human or animal subject,
means for processing said characteristic information in an information system to determine parameters of said deferred-use protocol, and
means for storing said processed information in a cell management data base.
